(19) United States
(12) Patent Application Publication
(10) Pub. No.: US 2005/0079516 A1
(43) Pub. Date: Apr. 14, 2005

(54) Chromosome stability assay

(75) Inventors: Andrew Maniotis, Berwyn, IL (US); Robert Folberg, Northbrook, IL (US)

Correspondence Address: David L. Parker;Fulbright & Jaworski L. L. P., 600 Congress Avenue, Suite 2400, Austin, TX, 78701, US

(73) Assignee: The Board of Trustees of the University of Illinois

(21) Appl. No.: 10/862,235

(22) Filed: Jun. 7, 2004

Related U.S. Application Data
(60) Provisional application No. 60/476,580, filed on Jun. 6, 2003.
(60) Provisional application No. 60/511,543, filed on Oct. 14, 2003.
(60) Provisional application No. 60/526,792, filed on Dec. 4, 2003.
(60) Provisional application No. 60/574,437, filed on May 26, 2004.

(51) Int. Cl. C12Q 1/68

(52) U.S. Cl. 435/6

(57) ABSTRACT

Embodiments of the invention relate to compositions and methods for evaluating or estimating the invasive potential of cells and thereby differentiating between normal and cancerous cells in accordance with the susceptibility of the cellular chromatin to degradation or other modification by particular enzymes or agents. Chromatin within permeabilized normal and non-invasive cells, including chromatin strands removed therefrom, are more susceptible to degradation by endonucleases or proteinases than is the chromatin from invasive cells.

This application claims priority to U.S. Provisional Patent applications Ser. No. 60/476,580, filed on Jun. 6, 2003; 60/511,543, filed Oct. 14, 2003; 60/526,792, filed Dec. 4, 2003; and serial number unknown, filed May 26, 2004; which are incorporated herein by reference in their entirety.

The government may own rights in the present invention pursuant to grant number RO1 EY10457 from the National Institutes of Health.

### BACKGROUND OF THE INVENTION

I. Field of the Invention

The invention relates to methods for detecting invasive mammalian cells and for differentiating between degrees of invasiveness of the cells. Specifically, the invention relates to methods for determining the sensitivity of chromatin to particular chromatin modifying agents, for example, the degradative action of the endonuclease ALU and/or the protease proteinase K, wherein the sensitivity of the chromatin to such agents is an indicator of the cancerous state and/or invasive potential of the cell.

II. Description of Related Art

Numerous methods have been devised for the detection of cancer. These range from the imaging of tumor masses by X-ray and optical techniques through the evaluation of cells in tissue samples obtained via biopsy to the detection of proteins and other molecular species that are expressed on the surfaces of cancerous cells or are released by cancerous cells into bodily fluids such as blood and urine. Once a cancer has been detected and localized, it is necessary to classify the cancer as to type and determine the characteristics of the cancer in order to arrive at an appropriate prognosis and treatment plan. An estimation of the invasiveness of the cancer cells is an important aspect of this characterization.

The detection, diagnosis, classification and characterization of cancers have traditionally been carried out through the visual microscopic evaluation of the morphologies of the cells comprising a tissue or cytological specimen. More recently, immunohistochemical and immunocytochemical methods for the detection and quantitation of certain cell surface proteins (markers) that are specifically or differentially expressed by cancerous cells have come into increasing use. Proteomic techniques that identify cancer cells by evaluating changes in the expression of large suites of proteins are under development, but are not yet in routine clinical use.

The evaluation of cellular morphology is generally considered to be the most definitive method for cancer detection, classification and characterization. Morphological evaluation is performed by specially trained, highly skilled personnel who examine differentially stained cell and tissue preparations on a cell by cell basis. This is a very labor intensive and somewhat subjective manual process that has a significant error rate due, in part, to the need to evaluate very large numbers of individual cells for the presence of any of numerous subtle morphological features. These morphological features and their interpretations can differ between types of cells and can be influenced by such factors as the medical history and demographics of the patient. Furthermore, normal repairative and reactive cellular processes often mimic the morphological changes observed in cancerous cells.

Automated image analysis systems for the evaluation of cell morphology, some of which are in clinical use, have been under active development for over fifty years. The utility of these systems for the detection, diagnosis and characterization of cancer cells is limited by the same factors that limit visual morphological evaluation as well as by factors such as the dynamic range of the image acquisition device that are unique to automated image capture and analysis systems. Image analysis systems in which morphological evaluation is combined with immunochemical staining methods such as described below are under development as a means of reducing analytical ambiguities, but have not yet been validated and accepted for widespread clinical use.

Immunohistochemical and immunocytochemical methods are based upon the observation that cancerous cells can express proteins that are not found in normal cells of that type or can express proteins that are found in normal cells at significantly higher concentrations or with different localizations than are found in normal cells. These proteins can be detected either qualitatively or quantitatively by means of immunological reagents that utilize antibodies that bind specifically to the target protein(s). In current practice, these immunological methods are primarily used to detect potentially abnormal cells, with the results being confirmed and refined using morphological methods.

A number of factors limit the utility of immunological methods for cancer detection, classification and characterization. Many tumors consist of mixtures of cell types, only some of which are cancerous and which may vary significantly in degree of invasiveness. Immunological reagents must therefore be capable of differentiating between the various cell types in such a mixture. Two critical steps in the development of an immunological method are the identification of a protein marker that allows differentiation between normal and cancerous cells and the generation of an antibody that specifically binds to this marker. Very few marker proteins that are truly unique to the cancerous state have been identified. Rather, the marker proteins that are known and are typically employed for cancer detection are normal cell constituents for which the amount, location and/or timing of expression differs between normal and cancerous cells. Thus rather than being able to differentiate between normal and cancerous cells in a binary manner on the basis of the presence or absence of marker expression, it is necessary to differentiate between normal and cancerous cells on the basis of semi-empirical assessment of relative staining intensities and localizations. Many of the immunochemical staining procedures presently employed for this purpose are not quantitative and, as is the case in morphological evaluation, it is not unusual for expression patterns of various markers in cancer cells to closely mimic those associated with normal proliferative cellular processes such as repair. These factors introduce additional ambiguity into such immunological evaluations.

The specificity of an antibody for its target analyte is another factor that has numerous implications for the utility of an immunological method. Different tumor types express different suites of markers and exhibit different marker expression patterns thus necessitating the use of different immunoreagents for each cancer type of interest. Unless a tumor is truly monoclonal, there will be some heterogeneity in the composition, structure and/or presentation in the target protein marker between cells. An extremely specific antibody may recognize only a subset of this heterogeneous mixture while a less specific antibody may recognize not only the various forms of the target marker, but related features of other markers as well. Furthermore, antibodies may bind non-specifically to locations on cells that are unrelated to the target marker. Target markers may also be masked in some manner and thus require “recovery” before they can be detected. These and numerous other factors that can limit the clinical utility of immunological methods are known to those skilled in the art.

Furthermore, there is a need to be able to objectively and accurately assess the invasive potential of cancer cells in order to be able to establish a prognosis and treatment plan. Current morphological and immunological methods provide an indication of invasive potential based upon empirical and qualitative correlations that have been established between certain morphological and immunological features and clinical outcome. For these and other reasons there is a need for an objective and unambiguous method for the detection of cancer cells that is generally applicable to a broad range of cancer types and that requires minimal interpretation in order to arrive at a clinically useful conclusion.

### SUMMARY OF THE INVENTION

For reasons discussed above there is a need for an objective and unambiguous method for the detection of cancer cells that is generally applicable to a broad range of cancer types and that requires minimal interpretation in order to arrive at a clinically useful conclusion. The present invention addresses this need by focusing upon a fundamental characteristic of all mammalian cells that underlies both the morphological and immunological features of these cells.

Furthermore, there is a need to be able to objectively and accurately assess the invasive potential of cancer cells in order to be able to establish a prognosis and treatment plan. Current morphological and immunological methods provide an indication of invasive potential based upon empirical and qualitative correlations that have been established between certain morphological and immunological features and clinical outcome. The present invention provides, typically, a quantitative method of estimating the invasive potential of cancer cells.

Embodiments of the invention relate to methods for evaluating or estimating the invasive potential of cells and thereby differentiating between normal and cancerous cells in accordance with the susceptibility of the cellular chromatin to degradation or other modification by particular enzymes or agents. In particular, the chromatin within permeabilized normal and non-invasive cells, and chromatin strands removed therefrom are more susceptible to modification by a chromatin modifying agent, e.g., degradation by the endonuclease ALU or the protease proteinase K, than is the chromatin from invasive cells. Furthermore, the chromatin within permeabilized normal cells is more susceptible to degradation by DNAase than is the chromatin within permeabilized invasive cells.

Certain embodiments of the invention include methods for assessing the invasive potential of a cell comprising contacting chromatin of a cell with one or more chromatin modifying agents and evaluating chromatin stability by assessing chromatin degradation. The methods may comprise isolating nuclei from the cells prior to contacting the chromatin with one or more chromatin modifying agents. The methods may further comprise isolating chromatin from the nuclei of the cells prior to contacting the chromatin with one or more chromatin modifying agents. In certain aspects, the cell, the nuclear membrane, or the cell and the nuclear membranes are permeabilized. A chromatin modifying agent may be a proteolytic enzyme, such as proteinase K; a nuclease; a DNAase; an endonuclease; or a combination thereof. In a preferred embodiment the endonuclease is ALU or MSP 1.

In other aspects, the chromatin may be re-aggregated after contacting with the chromatin modifying agent. In preferred embodiments, chromatin re-aggregation is initiated by contacting the chromatin with a DNA binding dye, a poly-amine, a histone, a topoisomerase, or glutaraldehyde prior to assessing chromatin stability. In further aspects of the invention, the evaluation of the chromatin is on a surface that is planar or approximately planar or is in a suspension in a fluid medium. In still further aspects, the chromatin degradation is evaluated qualitatively or quantitatively. In particular aspects, chromatin degradation is evaluated by visual microscopy, image analysis, or flow cytometry. Optical contrast of the chromatin may enhanced by contacting the chromatin with a DNA binding dye prior to evaluation. The DNA binding dye will typically comprise a chromatic dye or a fluorescent dye. The fluorescent dye may be ethidium bromide, acridine orange, TO-PRO, YO-YO, YO-PRO, PO-PRO or similar dyes known in the art.

A further embodiments of the invention include methods for assessing the effectiveness of a candidate therapeutic agent comprising contacting a cell with the candidate therapeutic agent; contacting the chromatin of the cell with a chromatin modifying agent as described herein; evaluating chromosome stability by assessing chromatin degradation as described herein; assessing the effectiveness of the candidate therapeutic agent by comparing chromatin degradation resulting from treatment of the cell with the candidate therapeutic agent with a cell not treated with the therapeutic agent. Still further embodiments of the invention are directed to therapeutic agent(s) identified by the processes of the invention.

In still further embodiments of the invention, the methods comprise differentiating between normal and cancerous cells by evaluating the degree of chromatin degradation by a chromatin modifying agent, preferably a nuclease, as describe herein.

Embodiments of the invention also include methods for detecting agents that can differentially degrade chromatin comprising contacting the chromatin with the agent being evaluated; evaluating the degree to which the chromatin is degraded, as described herein, by the agent being evaluated; determining the effectiveness of the candidate agent by assessing the differences in the degrees to which the chromatin of cells with differing degrees of invasiveness are degraded by the agent, as described herein.

It is contemplated that the methods or compositions described herein can be implemented with respect to other methods or compositions described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-1C. FIG. 1A shows a phase contrast image of chromatin strands removed from OCM-1a, M619 and MUM-2B melanoma cells. FIG. 1B shows a phase contrast image of the same chromatin strands after 30 minutes of treatment with ALU. FIG. 1C shows a fluorescence image of the same chromatin strands after 60 minutes of treatment with ALU followed by staining with ethidium bromide FIG. 2A-2I. FIG. 2A shows a phase contrast image of chromatin strands removed from normal human microvascular endothelial cells and from HT1080 fibrosarcoma cells. FIG. 2B shows a phase contrast image of the same chromatin strands shown in FIG. 2A after 1.25 hours of treatment with ALU. FIG. 2C shows a phase contrast image of the same chromatin strands shown in FIG. 2A after 2.25 hours of treatment with ALU. FIG. 2D shows a phase contrast image of chromatin strands removed from normal human microvascular endothelial cells and from HT1080 fibrosarcoma cells. FIG. 2E shows a phase contrast image of chromatin strands shown in FIG. 2D after 5 minutes of treatment with proteinase K. FIG. 2F shows a phase contrast image of chromatin strands shown in FIG. 2D after 5 minutes of treatment with proteinase K followed by treatment with the DNA aggregating agent polyamine 11172. FIG. 2G shows a phase contrast image of chromatin strands isolated from mesenchymal stem cells and HT1080 fibrosarcoma cells. FIG. 2H shows a phase contrast image of chromatin strands isolated from mesenchymal stem cells and HT1080 fibrosarcoma cells after 5 minute treatment with proteinase K. FIG. 21 shows a phase contrast image of chromatin strands isolated from mesenchymal stem cells and HT1080 fibrosarcoma cells after 5 minute treatment with proteinase K and condensation of the DNA with glutaraldehyde.

FIG. 3A-3D. FIG. 3A shows a phase contrast image of minimally invasive OCM-1a human melanoma cells. FIG. 3B shows a fluorescence image of the same cells as shown in FIG. 3A after permeabilization; treatment with ALU; and staining with ethidium bromide. FIG. 3C shows a phase contrast image of highly invasive MUM-2B human melanoma cells. FIG. 3D shows a fluorescence image of the same cells as shown in FIG. 3C after permeabilization; treatment with ALU; and staining with ethidium bromide.

FIG. 4A-4C. Studies showing the sensitivities of fibroblasts (FIG. 4A), OCM 1a (FIG. 4B)(poorly invasive melanomas), and MUM 2B (FIG. 4C)(highly invasive melanomas) after 24 hours of incubation with MSP I. Note that fibroblast nuclei are completely digested in 24 hours. OCM 1a nuclei showed some focal residual staining, while MUM 2B nuclei exhibited complete stability and sequestration from the methylation-specific enzyme.

FIG. 5 shows flow cytometer fluorescence intensity histogram plots measured for each of WI-38 fibroblasts (normal cells); OCM1 (a poorly invasive a primary uveal melanoma); M619 (a highly invasive primary uveal melanoma; and MUM2B (a highly invasive metastatic uveal melanoma) at 1, 3 and, 5 hours exposure to Alu I restriction enzyme followed by staining with PI.

FIG. 6 shows forward scatter (representing cell size), and side scatter (representing internal cellular complexity) before exposure to Alu I and after 1, 3, and hours digestion for each of the four cell lines WI-38 fibroblasts (normal cells); OCM1 (a poorly invasive a primary uveal melanoma); M619 (a highly invasive primary uveal melanoma; and MUM2B (a highly invasive metastatic uveal melanoma).

### DESCRIPTION OF ILLUSTRATIVE EMBODIMENTS

Chromatin structure is an important factor in many aspects of cell regulation and function including genetic transcription, replication and recombination. Although the detailed mechanisms that control higher order chromatin structure and its changes during the cell cycle are still poorly understood, the gross aspects of chromatin structure can be correlated with cell status and are widely used as major elements in the morphological differentiation between normal and cancer cells. Furthermore, expression of the genes that comprise the chromatin results in the generation of the immunological and other morphological features that are used for this purpose. The relationships between chromatin structure, gene expression and morphological and immunological features is an area of active research.

The susceptibility of chromatin to modification, such as degradation, by external agents, such as nucleases, endonucleases and proteases, is a function of chromatin structure. The term “external agents” refers to agents other than the nucleic acids, histones and other materials that are part of the chromatin structure. “Chromatin modifying agents,” as used herein, refers to agents that differentially effect chromatin associated with normal and cancerous cells. Differential effects may be manifest as differences in the sensitivity of chromatin to degradation, for example by a nuclease or an endonuclease or a protease. The present invention is based upon the observation that certain chromatin modifying agents, e.g., specific endonucleases, nucleases, proteases and other chemicals or compounds, may result in degradation of chromatin in a unique manner that beneficially relates this observation to the assessment of a cancerous state and/or invasive potential of the cell.

The invention relates to methods for estimating or assessing the invasive potential of cells and/or, more generally for differentiating between cancerous and non-cancerous (normal) cells.

The chromatin that comprises the entire genome of a cell consists of double stranded DNA that is at least partially encased in proteins such as histones that modulate gene expression. Exposure of this DNA to external agents such as transcription factors and endonucleases is determined by or determines what genes are being expressed at any given time. As a consequence, only certain portions of the DNA comprising the genome of a cell are exposed to external agents in normal cells. This pattern of exposure changes when a cell becomes cancerous and changes further depending upon the degree of invasiveness of a cancer cell. As a consequence, the susceptibility of a chromatin strand to degradation by a chromatin modifying agent varies in a systematic manner depending upon whether the cell containing the chromatin was normal or cancerous and, if cancerous, upon the degree of invasiveness of the cancer. The susceptibility of the proteins that encase the chromatin DNA to degradation by proteolysis similarly varies depending upon whether the cell is normal or cancerous.

Assessment or evaluation of the susceptibility of the chromatin of a cell to degradation by a chromatin modifying agent, e.g., a nuclease, an endonuclease or a protease, therefore permits determination of whether the cell was normal or cancerous and, if cancerous, the degree of invasiveness of the cancer. Such an assessment can be carried out in a qualitative, semi-quantitative or quantitative manner for the purposes of detecting and staging cancers. Embodiments of the invention include chromatin strands that have been removed from a cell.

I. Methods Related to Chromatin Stability Assay

Embodiments of the invention may be used to evaluate one or more cells for a pre-cancerous, cancerous and/or invasive character. Suitable samples for use in the present methods include, but are not limited to cultured cells and to cells obtained from tumor biopsies, other tissues, organs and cell-containing bodily fluids from a subject or patient.

Normal endothelial cells and melanoma cell lines including, but not limited to OCM-1a (non-invasive); M619 (invasive); and MUM-2B (metastatic, highly invasive) are representative of some of the cell types that may be used in the practice of this invention. These and similar cell types will be will be used in the illustration of certain embodiments of this invention.

A. Methods Utilizing Isolated Chromatin

Assessment of chromatin stability may be performed using chromatin isolated from normal and/or cancerous cells. The chromatin that comprises the entire genome of a cell may be microsurgically removed from a cell and externally manipulated as a single chromatin strand in accordance with published techniques. Cells from which the chromatin is to be microsurgically removed are most conveniently prepared by growing the cells to near confluence on a solid substrate. This process results in the cells forming strong attachments to the substrate and prevents them from moving in response to the mechanical forces that are applied to the cells during the microsurgical procedure. This anchoring of cells to a solid substrate is for operational convenience only and is not essential to the practice of the present invention. Adherent cells suitable for the microsurgical extraction of chromatin can be prepared by methods such as are described in Example 1: Materials and Methods (below). The specific method described in Example 1 is primarily applicable to endothelial, melanoma cells, and other cell types derived from endothelial cells. This method can be adapted to other cell types by means of adjustments to the composition of the growth medium and related parameters in manners that are known to those skilled in the art.

The microsurgical extraction of cellular chromatin is most conveniently performed on metaphase cells having well centered and condensed mitotic plates, but can be performed on cells that are in any phase of the cell cycle. A glass micropipette having a tip diameter in the range of one to five microns and a bore in the tip of less than 0.5 microns can be used to rapidly pierce (“harpoon”) the cell nuclear membrane. Bringing the tip of the micropipette into contact with the chromatin causes the chromatin to adhere to the tip of the micropipette via non-covalent forces. A weak suction can be applied to further enhance the adhesion of the chromatin to the tip of the micropipette. Withdrawing the micropipette extracts the chromatin from the nucleus. When this microsurgical extraction procedure is applied to mitotic cells in telophase, only one set of daughter chromatin is removed from the nucleus. Applying this procedure to cells in interphase or to mitotic cells in prophase, metaphase or anaphase results in the removal of all chromatin from the nucleus. Alternatively, the cell can be induced to eject its chromatin by using the tip of the micropipette to rupture the nuclear and cytoplasmic membranes of the cell. The ejected chromatin strand(s) may then be anchored to the tip of the micropipette as previously described.

The micropipette can be used to manipulate the attached chromatin strand(s). One such manipulation consists of the transfer of the chromatin strand(s) to a glass or plastic substrate other than the one upon which the cells are attached and the arrangement of the strand(s) as desired upon said substrate in preparation for evaluation by methods such as are described below. As exposure of the chromatin to air can cause irreversible damage to the chromatin, it is preferable that the substrate to which the cells are attached and the substrate to which the chromatin is transferred reside in the same pool of fluid medium and that all manipulations take place under the surface of this medium. One convenient implementation is to adjacently place the substrate to which the cells are attached and the substrate to which the chromatin is to be transferred in the same plastic culture dish prior to performing the microsurgical extraction of the chromatin. An alternative implementation is to use the inner surface of the plastic culture dish as the substrate to which the chromatin strands are transferred. Upon contact with the receiving substrate, the chromatin strand adheres to said substrate via non-covalent forces. Multiple chromatin strands from the same and/or different cell types may be placed at separate locations on a single substrate. One preferred configuration places multiple chromatin strands isolated from each of several cell types on a single substrate. This configuration facilitates redundant comparisons between the chromatin strands from the different sources. Other arrangements may be used as desired and/or appropriate.

Suitable substrates to receive the chromatin strands include, but are not limited to glass such as a glass cover-slips or a plastic such as polystyrene. These substrates may, but are not required to be, coated or treated to promote adhesion of the chromatin strand to the substrate. Some suitable adhesion promoting coatings include, but are not limited to gelatin; serum proteins, matrix proteins such as fibronectin, a polyamine such as poly-lysine; or a poly-aminosilane. Plastic substrates may also be treated using a gas plasma, corona or glow discharge to introduce oxy- and/or amine functionality into the surface of the plastic. These and other such methods of adhesion promotion are well known to those skilled in the art.

The microsurgical extraction and manipulation of chromatin strands is preferably performed in a medium of low ionic strength (30-55 mM) and containing approximately 2 mM Mg⁺⁺ ions such as is described below in order to maximally preserve the chromatin compaction and the retention of proteins that are found in association with the chromatin. Subsequent enzymatic treatments of the chromatin strands are, however, preferably carried out under physiological or near physiological conditions of ionic strength in order to optimize the activity of the enzyme(s) employed. For this reason, it is desirable to increase the ionic strength of the medium from approximately 30 to 55 mM to approximately 0.15 M by the addition of NaCl after the chromatin strands are adhered to the receiving substrate.

The described methods of specimen preparation are broadly applicable, but aspects such as, but not limited to, the use of primary or cultured cells, the identified cell culture conditions and microsurgical techniques may be selected and modified as appropriate to the sample or specific cell type(s) from which specimens are being prepared. Such modifications are known to those skilled in the art and are not limiting to the scope of the present invention.

B. Chromatin Stability Assay in Cells

The methods of the invention may be applied to intact cells as well as to isolated chromatin strands. By way of example, intact cells of interest may be located, placed or grown on a solid support, such as a glass cover slip, coated with absorbed serum proteins and can be permeabilized by treatment with a detergent, e.g., Triton X-100; washed to remove residual detergent; and treated with an appropriate chromatin modifying agent. Embodiments of the invention may be practiced using living or preserved cells adherent to a supporting substrate and to living and preserved cells that are suspended in a medium. In certain embodiments, the nuclei of a cell of interest may be isolated prior to performing chromatin stability assays. A quantitative chromosome stability assay is based upon the susceptibility of chromatin to digestion by certain endonucleases, nucleases and proteases, which reflect the degree of invasiveness of the cell containing or providing the chromatin.

In certain aspects of the invention, methylation may generally increase at the level of higher order chromatin structure throughout the genomes of more invasive cells. Typically, methylation of specific genes using MSP PCR is detected with a range of molecular “kits” available from a variety of companies, for example, Serologicals Corporation (Norcross, Ga.), OncoMethylome Sciences S.A. (Durham, N.C.), and others. Qiagen (Valencia, Calif.), for example, has developed MSP PCR to employ methylation-specific PCR for several specific promotors. Methylation-specific PCR of these promoters allows mapping of DNA methylation patterns in GC-rich regions of DNA. It is assumed that hypermethylation of promoter regions is often a decisive factor in inactivation of tumor suppressor genes in human cancers.

Because of these difficulties, an assay has been developed by the inventors that employs chromatin testing of populations of cells under normal physiological ionic conditions in lysed cell models, in assays that employ flow cytometry, and in smear preps similar to Pap smears. The test is based upon the cell as an integrated mechanical unit whose genetic sequestration and exposure is controlled not only from the level of histone octamers or topoisomerases (Maniotis et al., 1997; Bojanowski et al., 1998), but at the level of higher order chromatin structure (Garinis et al., 2002; Chen et al., 2003). By testing the sensitivity of Alu, Eco RI, Mbo, Hind-1, PST-1, and other specific and non-specific nucleases and proteases, the inventors have determined that disulfide-rich proteins differentially sequester Alu sequences as cells increase their invasive behavior.

MSP I digestion sensitivity or digestion insensitivity was tested as a generalized property of nuclei within cells of increasing invasive and malignant behavior. The results of these studies show that sequestration and exposure of methylated sites occurs at the level of higher order chromatin folding, and not only at the level of specific putative cancer genes or gene sequences.

II. Cell Preparation from Tissues

Cells can be isolated from tissues such as tumor biopsies by methods known to those skilled in the art (for a general review see Freshney, 1987). Such methods are generally similar to those described for the isolation of extracellular matrix protein from liver except that the tissue may be incubated with or homogenized in a medium that contains proteolytic enzymes such as trypsin to disrupt cell-cell interactions and that the desired cells are found in the cellular pellet rather than the supernatant.

Cell culture can be performed in accordance with methods known to those skilled in the art. In most instances, a suitable growth medium consists of DMEM (BioWhittaker, Walkersville, Md.) supplemented with 10% fetal calf serum and, where relevant, suitable concentrations of cell growth factors such as, but not limited to basic fibroblast growth factor, transforming growth factor β, vascular epithelial growth factors, interleukins and other such agents as may be required for the proper growth of the particular cell type(s) being cultured. In certain embodiments antibacterial or antifungal agents are not used in the culturing of cells for use in the practice of this invention as such agents are known to interfere with the differentiative potential of primary cell types. Cell culture is performed at 37° C. under an atmosphere consisting of approximately 5% CO₂/balance air.

III. Data Capture & Interpretation

Nucleic acid staining in cells of interest can be monitored visually and/or can be captured as electronic images for subsequent quantitative analysis by means of microscopic imaging that are well known to those skilled in the art, for general methods see Current Protocols in Cell Biology (2001); or Murphy, Fundamentals of Light Microscopy and Electronic Imaging (2001). One suitable microscopy platform for visual imaging and electronic image capture consists of a Leica DM IRB inverted microscope (Leica, Wetzlar, Germany Microsystems Inc., Bannockburn, Ill.) equipped for transmitted light, phase contrast, differential interference contrast and epi-fluorescence visual and electronic imaging at magnifications of ×20, ×40, and ×63. This microscopy platform may also equipped with means to maintain the specimen being imaged at any desired temperature, most commonly about 25° C. or 37° C., to facilitate the monitoring of the time courses of the reactions over extended periods of time. A comparably equipped upright microscope such as a Leica model LS or LB (Leica Microsystems Inc., Bannockburn, Ill.) may also be employed.

Images of the specimens can be captured electronically by means of a CCD video camera, or similar apparatus, with or without an image intensifier and stored electronically in computer memory, and/or magnetic or, optical or other information storage media such as CD-ROM or video tape. Other means of image capture and storage may also be employed. Electronically captured images can be evaluated utilizing image analysis methods that are well known to those skilled in the art, see Current Protocols in Cytometry (1997) or Digital Image Processing: PIKS Inside (2001) for general methodology. For example, differentiation between cells in which the chromatin has and has not been digested by DNAse and fluorescently stained may be accomplished by utilizing an adaptive thresholding method to segment the image into regions exhibiting pixel intensities above (putative nuclei) and below a threshold value and subsequently determining the size, shape and mean or integrated pixel intensity of each above threshold region. One convenient image analysis method determines and evaluates the image signal level at each pixel location as a function of time and computes the mean pixel signal levels within defined regions of interest along a chromosome.

One of many possible suitable embodiments of such a method utilizes a DAGE MTI (Michigan City, Ind.) or a Photometrics (Tucson, Ariz.) cooled CCD camera to capture images of the specimen. Automatic image focusing is accomplished using the constrained iterative autofocus algorithm included in the VayTek Microtome image deconvolution software package (VayTek, Fairfield, Iowa). Regions of interest can be manually defined and the mean pixel signal levels within these regions can be determined using the Scanalytics IPLab image quantitation software (Scanalytics, Fairfax, Va.). This software can also be employed to perform routine image preparation operations including, but not limited to field flattening; background and “hot pixel” correction; and fixed and/or adaptive thresholding. More sophisticated methods that are known to those skilled in the art such as, but not limited to pixel tracking; morphological analysis; pattern matching; correlation and similar algorithmic image analysis methods may be beneficially employed as appropriate to specific applications of the present invention. Embodiments of the invention contemplate the automation of the methods described herein. Various steps in the methods and processes described herein are amenable to automation as is known to those skilled in the art.

The presence of exogenous materials such as stains that are commonly utilized to facilitate the visibility of cells, cell constituents, cell structures, and isolated chromatin in transmitted light, reflected light and fluorescence microscopic techniques can potentially interfere with the isolation, manipulation and digestion of chromatin. For this reason, certain embodiments it is beneficial to utilize phase contrast or other similar imaging modalities that do not require the use of such contrast enhancement agents to facilitate specimen visualization and/or imaging until such time as cell chromatin modification, e.g., digestion, has been completed. After chromatin modification has been completed at which time, the specimen may be treated with a DNA binding dye, stain or other reagent that selectively increases the contrast between the chromatin and the other materials in the specimen. For example, the fluorescent DNA binding dye ethidium bromide is specified in the following descriptions of preferred embodiments of the invention. Numerous additional suitable fluorescent, absorbing and other types of contrast enhancement agents are known to those skilled in the art, see for example Molecular Probes: Handbook, updated Sep. 7, 2003, probes.com/handbook, which is incorporated herein by reference. Of these, certain fluorescent DNA binding dyes including, but not limited to dyes of the TO-PRO, YO-YO, YO-PRO and PO-PRO families, (Molecular Probes, Eugene Oreg.), that bind specifically and stoichiometrically to DNA and that undergo a significant enhancement in fluorescence upon binding to DNA are particularly beneficial in those certain embodiments of the present invention wherein it is desired to quantitate the amount of DNA present. Fluorescent DNA binding dyes are preferred when quantitation of the amount of DNA present is desired. Many DNA binding dyes including, but not limited to those cited above, are capable of condensing and thus improving the visibility of de-condensed chromatin.

Numerous other suitable methods of microscopic imaging, image capture, and image analysis are known to those skilled in the art. The methods identified herein are for exemplary purposes and do not in any way limit or constrain the scope of the present invention.

One method for analyzing the staining and assessment of cellular DNA is by flow cytometry or laser scanning cytometry. In an even more preferred embodiment, cells that are stained with a quantitative DNA stain are subjected to flow cytometry. Flow cytometry can be performed with a fluorescent activated cell sorter (FACS) as known in the art. Exemplary FACS machines that can be used include FACS-Calibur (Becton Dickinson; Mountain View, Calif.) and a Coulter flow cytometer (Hialeah, Fla., USA) EPICS Elite®. Quantification can be performed using CellQuest (Becton Dickinson; Mountain View, Calif.), WinList (Verity Software House, Inc. Topsham, Me.), Multicycle software (Phoenix Flow Systems, San Diego, Calif. USA) and FACScan (Becton Dickinson, Mountain View, Calif.) software.

A flow cytometer measures the amount of light-emitting substance associated with each cell and other parameters and provides output in the form of, e.g., a histogram, dot plot, or fraction table. The amount of one light-emitting substance associated with each cell can be compared to other properties of that cell, such as the amount of another light-emitting substance to which the cell population has also been exposed, size, granularity, or inherent light-emission.

As sheath fluid containing cells passes through the laser, typically one-by-one, they are exposed to light of various wavelengths. Each particle detected by the cytometer is termed an “event.” The degree to which an event transmits or scatters some of the incident light provides a measure of the event's characteristics, e.g., associated light emitting substance. For example, the event may emit light of its own accord or may emit fluorescent light generated by a fluorescent substance introduced into the event. An example of such a substance is a fluorescent DNA stain. A fluorophore responds to incident light of a particular frequency by emitting light at a known frequency that is detected by, e.g., photomultiplier tubes (PMTs) of the cytometer. The intensity of the emitted or reflected light is measured and stored by the cytometer.

The cytometer compiles emission data into a histogram. The histogram may be reported in one-dimensional form. Alternatively, it may be combined with a histogram of emitted light resulting from other incident wavelengths. Such a combination is typically reported as a “dot plot,” in which events are plotted on a grid, and the axes of the grid correspond to the two parameters being measured. For example, events could be exposed to incident light of a particular wavelength and assayed for forward light scatter and for emission at another wavelength.

A cell population may be segregated based on their DNA content. A peak will occur at propidium iodide (PI) staining corresponding to the normal DNA content of cells. Peaks may also occur at higher multiples of the haploid number n, possibly corresponding to polyploid or mitotic cells. Peaks or above-background plateaus may also occur at PI staining levels that do not correspond to multiples of haploid number n. These events may correspond to cells that are sensitive to various DNA degradative agents. Gates may be formed to distinguish cells falling into various ranges of DNA content from cells with differing DNA content.

Other methods for identifying the DNA content of cells using a quantitative DNA stain and histochemistry. These techniques can also be combined with flow cytometric analysis. For example, certain cells can be separated out from other cells via flow cytometry. These cells can then be analyzed for DNA content using a non flow cytometric techniques.

Chromatin condensation can also be achieved through the use of non-staining reagents including, but not limited to histones such as histone H1; topoisomerases such as topoisomerase I and II; a polyamine such as polyamine 11172 and polyamine 11158; DNA crosslinking agents such as gluteraldehyde; and changes in the ionic strength and composition of the medium surrounding the chromatin.

Numerous other suitable methods of microscopic imaging, image capture and image analysis are known to those skilled in the art. In certain embodiments, flow cytometry may be used in evaluating processed cells and/or nuclei in accordance with methods known to those skilled in the art. The methods identified herein are intended only for illustrative purposes and do not in any way define or constrain the scope of the present invention.

IV. Chromatin Modifying Agents

Embodiments of the invention utilize certain chromatin modifying agents that differentially modify or act on chromatin of a non-invasive cell (normal cell) versus chromatin of a cancerous cell, in particular a cancerous cell with an invasive phenotype.

A. Nucleases

Endonucleases comprise a large class of enzymes that as their primary function cleave DNA strands at specific locations. Some endonucleases cleave DNA strands only at sites defined by very specific combinations of nucleic acid sequence and DNA conformation while others are less demanding in the characteristics of the sites at which they cleave DNA. In any case, however, the DNA must be accessible to the endonuclease in order for cleavage to occur. The number and identity of the locations along a chromatin strand at which the DNA component of the chromatin is exposed to external agents is determined by the status of the cell from which the chromatin was obtained. The cleavage of chromatin by an endonuclease occurs only at those locations where the nucleic acid sequence and physical conformation of a segment of exposed DNA corresponds to the specificity of the endonuclease. The pattern of DNA strand cleavage obtained when a chromatin strand is treated with any particular endonuclease is therefore a proxy for the structure of the particular chromatin strand. This pattern can be evaluated explicitly by known methods such as gel electrophoresis or implicitly by the methods of the present invention.

Chromatin exposed to the action of the endonuclease ALU or MSP 1 or, alternatively, the action of the nuclease DNAase, is degraded in a manner and to a degree that correlates with the invasive potential of the cell from which the chromatin was derived. The degradation of chromatin with HIND III, BAM, EMBO, PST I, SAU-I, RNase A, RNase I or micrococcal nuclease does not correlate with the invasive potential of the cell from which the chromatin was obtained. However, other nucleases and endonucleases that do exhibit a differential chromatin cleavage pattern can be identified by the methods described herein.

B. Proteinase

Proteins such as histone H1 are known to play a critical role in chromatin organization. It is, for example, known that the treatment of chromatin from a normal cell with an agent such as proteinase K (50 ug/ml) or heparin (5 mg/ml), both of which are known to remove proteins from chromatin, result in the rapid decondensation of the chromatin into a diffuse cloud of DNA. This decondensation cannot be reversed by increasing or decreasing the ionic strength or Mg⁺⁺ concentration in the surrounding medium, but is essentially completely reversed with reconstitution of the original chromatin morphology by the addition of histone H1. This reconstitution also restores the ability of the chromatin to condense and/or recondense in response to changes in ionic strength and Mg⁺⁺ concentration.

One aspect of the present invention is that the effects of removing proteins from chromatin strands obtained from abnormal and invasive cells through the use of the protease proteinase K differ in a novel and useful manner from the effects observed when chromatin strands from normal cells are treated with proteinase K and from the effects observed when chromatin strands from normal and abnormal cells are treated with other agents including, but not limited to the protease trypsin or protein binding modulators such as heparin, sodium dodecylsulfate, mercaptoethanol and/or dithiothreitol.

V. Methods of Screening Candidate Substances

Assays based upon the present invention may be used to differentiate between invasive and non-invasive cells; to detect and evaluate substances that can modulate or otherwise alter the invasiveness of a cell; and to detect and evaluate substances that can differentially degrade chromatin in a manner that is dependent upon the invasiveness of the cell from which the chromatin was obtained.

Assays that differentiate between invasive and non-invasive or normal cells find utility in applications such as the characterization of a cancer as part of the development of a prognosis and treatment plan. An assay for such purposes may be structured as follows: Cancerous cells are isolated from a tumor or other cancer in accordance with standard methods. In similar manner, normal or non-cancerous cells of the same or similar type are obtained from the same or related tissue to serve as a control. Cultured invasive cells of a similar type may be used as another control. The chromatin is isolated from both the cancerous and control cells as described in Section IA; treated with ALU or another agent as described in Section IB; treated with a DNA stain such as ethidium bromide to facilitate visualization; and the results evaluated either visually or through use of the methods described in Section II. The invasiveness of the cells isolated from the cancer is inversely proportional to the degree to which the chromatin is degraded in this assay.

Assays for the detection and evaluation of substances that can modulate or otherwise alter the invasiveness of a cell are of primary utility in the screening of new chemical entities (NCEs) and other substances for the purpose of identifying potential anti-cancer therapeutic agents. Such assays may also be of use in determining the efficacy of a particular therapy against a specific cancer in the context of planning a therapeutic regimen or monitoring the effectiveness of a therapy. This same assay format may be used to detect agents that promote the formation or invasiveness of cancers for purposes such as environmental monitoring. Such assays may be structured in the following manner: Primary or cultured cells of the cancer type of interest and normal control cells of the corresponding type are obtained from sources appropriate to the intended application. One set of these cells is treated with the substance under test in accordance with an appropriate protocol. A second set of these cells is left untreated with the substance in order to serve as a control. The chromatin is isolated from both the cancerous and control cells as described in Section IA; treated with ALU or another agent as described in Section 11B; treated with a DNA stain such as ethidium bromide to facilitate visualization; and the results evaluated either visually or through use of the methods described in Section II. The efficacy of an substance in reducing or blocking the invasiveness of a cancer is evidenced by increased degradation of the chromatin from cells treated with the substance under test relative to the corresponding control cells. If this assay is being performed in order to detect substances that promote invasiveness, the experimental cells are typically ones that are normal or of low invasiveness and the presence of a promoting substance is evidenced by a decrease in chromatin degradation relative to the corresponding controls.

If the assay is intended for the detection of substances that differentially degrade chromatin in accordance with the degree of invasiveness of the cells from which the chromatin is obtained, the chromatin used in the assay is obtained from cells having different known degrees of invasiveness. The chromatin is isolated from these cells as described in Section IA and is divided into two groups. The experimental group is treated with the substance under test while one control group is treated with ALU or another known differentially acting agent as described in Section IB and a second control group is left untreated. The chromatin from these groups is treated with a DNA stain such as ethidium bromide to facilitate visualization; and the results evaluated either visually or through use of the methods described in Section II. Differential degradation is evidenced by chromatin from experimental cells of one or more levels of invasiveness being more completely degraded than is the chromatin of the other cells in the experimental group. Assays of this type are of utility in detecting, identifying and characterizing differentially acting substances that are more specific and/or more potent than ALU and other known differentially acting substances.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of Isolated Chromatin Strands

The isolated chromatin strands utilized as specimens in certain embodiments of the present invention can be prepared by the general methods described above and the specific methods as follows.

Cultured cells from which the chromatin is to be microsurgically removed are most conveniently prepared by growing the cells to near confluence on a solid substrate. Similarly, primary cells obtained from a tissue specimen or other source may be dispersed and allowed to attach to a suitable solid substrate in accordance with standard methods. This process results in the cells forming strong attachments to the substrate and prevents them from moving in response to the mechanical forces that are applied to the cells during the microsurgical procedure. This anchoring of cells to a solid substrate is for operational convenience only and is not essential to the practice of the present invention. In the present examples, adherent human melanoma and endothelial cells suitable for the microsurgical extraction of chromatin can be prepared by culturing the cells to near confluence on gelatin-coated glass cover-slips in complete medium containing DMEM, 10% calf serum and 25 mM Hepes buffer at pH 7.4; transferring the cover-slips to which cells are adhered into a 35 mm plastic culture dish containing approximately 2 mL of DMEM that has been buffered to pH. 7.4 with Hepes and that contains approximately 30-55 mM of NaCl and approximately 2 mM of MgCl₂ (the medium); and allowing these preparations to equilibrate at 37° C. in an atmosphere of approximately 10% CO₂/balance air prior to microsurgery.

The microsurgical extraction of cellular chromatin is most conveniently performed on metaphase cells having well centered and condensed mitotic plates, but can be performed on cells that are in any phase of the cell cycle. A glass micropipette having a tip diameter in the range of one to five microns and a bore in the tip of less than 0.5 microns can be used to rapidly pierce (“harpoon”) the cell nuclear membrane. Bringing the tip of the micropipette into contact with the chromatin causes the chromatin to adhere to the tip of the micropipette via non-covalent forces. A weak suction can be applied to further enhance the adhesion of the chromatin to the tip of the micropipette. Withdrawing the micropipette extracts the chromatin from the nucleus as a single strand. When this microsurgical extraction procedure is applied to mitotic cells in telophase, only one set of daughter chromatin is removed from the nucleus. Applying this procedure to cells in interphase or to mitotic cells in prophase, metaphase or anaphase results in the removal of all chromatin from the nucleus as a single strand. Alternatively, the cell can be induced to eject its chromatin by using the tip of the micropipette to rupture the nuclear and cytoplasmic membranes of the cell. The ejected chromatin strand may then be anchored to the tip of the micropipette as previously described.

### Example 2

### Nuclease Sensitivity

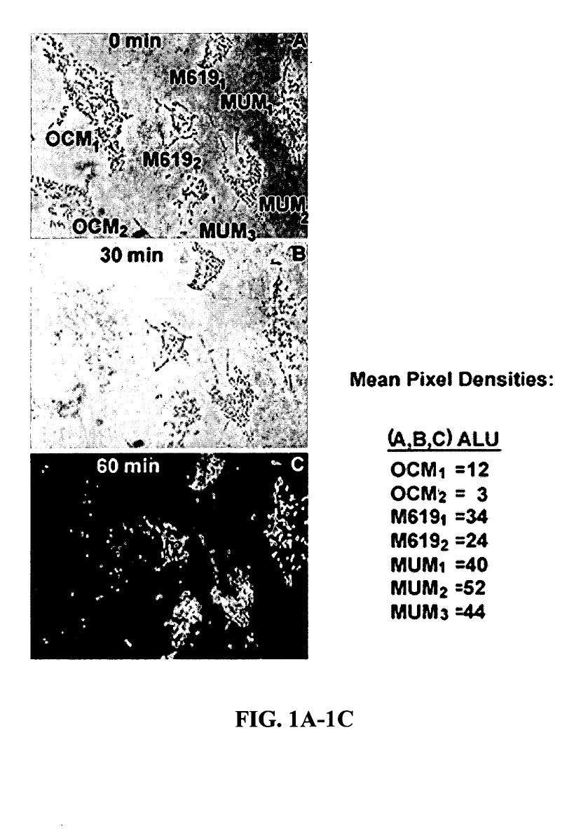

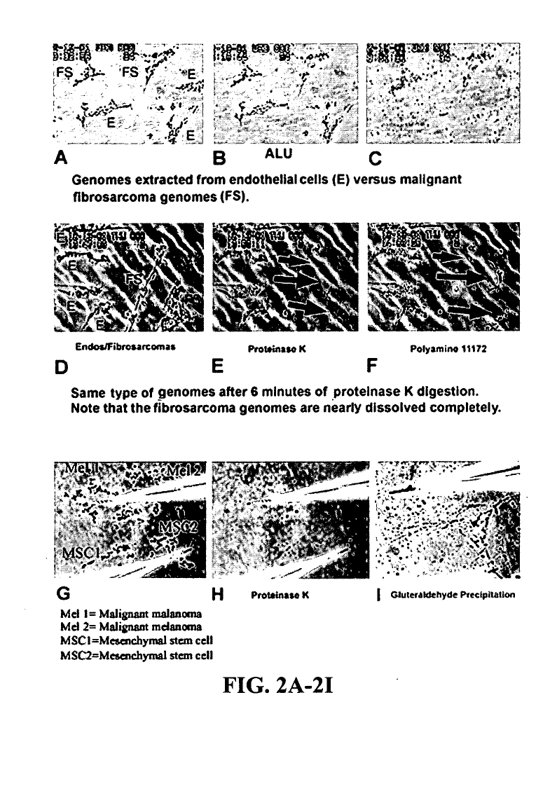

FIG. 1A shows chromatin strands isolated from the cells of the OCM-1a, M619 and MUM-2B melanoma cell lines adhered to a glass substrate as described above and imaged in phase contrast. OCM-1a, M619 and MUM-2B are known by independent means to be non-invasive, invasive (primary), and highly invasive (metastatic) melanoma cell lines, respectively. FIG. 1B shows the same chromatin strands viewed in phase contrast 30 minutes after 5 units of the endonuclease ALU was added to the fluid medium surrounding the chromatin strands while FIG. 1C shows a fluorescence image of these same chromatin strands 60 minutes after the addition of ALU and subsequent staining of the chromatin with ethidium bromide. Ethidium bromide is a fluorescent dye that binds specifically to DNA and causes the DNA to which it is bound to condense and precipitate. It can be seen in the examination of these three images that the chromatin obtained from the non-invasive OCM-1a melanoma cell line is essentially completely degraded by treatment with ALU while the chromatin from the invasive M619 and highly invasive MUM-2B cell lines is largely unaffected by this treatment.

The amount of DNA present in each chromatin strand can be quantitated by methods previously described. When normalized to the amount of DNA initially present in each chromatin strand, it can be determined that less than 10% of the chromatin from highly invasive MUM-2B was degraded during a 60 minute treatment with ALU under the indicated conditions while approximately 30% of the chromatin from invasive M619 cells and over 85% of the chromatin from non-invasive OCM-1a cells were degraded under the same conditions. Chromatin strands isolated from normal epithelial cells (not shown) are essentially completely degraded under the same conditions.

FIG. 1 illustrates that the chromatin isolated from normal human endothelial cells and non-invasive human melanoma cells is more extensively degraded by ALU than is the chromatin from invasive and highly invasive human melanoma cell types. This same pattern of behavior in which the chromatin isolated from invasive cells is more resistant to degradation by ALU than is chromatin from normal and non-invasive cells is consistently observed in all human cell types evaluated and in cell types from all other mammalian species that have been examined. Furthermore, the transfection of normal cells by the insertion of one, two or three additional genes progressively increases the resistance of the resulting chromatin to digestion by ALU with the chromatin from normal cells being most strongly degraded and that from the triply transfected cells being the least degraded.

The sensitivity of the chromatin strands to digestion by ALU is not correlated with the ploidy of the cell from which the chromatin was extracted. Non-invasive OCM-1a cells are, by way of example, well known to be near triploid whereas highly invasive MUM-2B cells are known to vary from near diploid to polyploid. In all cases, however, the chromatin from triploid OCM-1a cells is more sensitive to digestion by ALU than is the chromatin from diploid or polyploid MUM-2B cells. Furthermore, chromatin strands from diploid and polyploid MUM-2B cells are equally resistant to digestion by ALU.

Degradation of chromatin strands with other endonucleases including, but not limited to HIND III, BAM, EMBO, PST-1, SAU-1, RNase A, RNase 1, and micrococcal nuclease does not exhibit chromatin cleavage patterns that correlate with the invasive potential of the cell from which the chromatin was obtained.

### Example 3

### Chromatin Condensation or Re-Aggregation

The effects of proteinase K on chromatin isolated from non-invasive and invasive cell types are illustrated in FIG. 2. FIGS. 2A, B and C, which are presented for reference purposes, illustrate the effects of treatment of chromatin strands from normal human microvascular endothelial cells and invasive HT1080 fibrosarcoma cells with ALU in the manner described above. The chromatin from normal endothelial cells is extensively degraded while that from the fibrosarcoma remains largely intact.

FIGS. 2D and 2E illustrate the effects of treatment of chromatin strands from normal human microvascular endothelial cells and invasive HT1080 fibrosarcoma cells with proteinase K under the conditions described above except that proteinase K (5 mg/ml) was substituted for ALU and the incubation time was reduced from 60 minutes to 5 minutes. The chromatin from the fibrosarcoma cells was extensively dispersed while that from the normal the endothelial cells remained largely intact. Furthermore, as is illustrated in FIG. 2F, treatment of the proteinase K digested chromatin strands with polyamine 11172, an agent that is known to be able to recondense dispersed chromatin, largely recondenses the endothelial cell chromatin while little if any recondensation of the protease treated fibrosarcoma chromatin occurs.

The effects of proteinase K treatment of chromatin strands from normal rat mesenchymal stem cells and invasive human metaphase M619 melanoma cells followed by the non-specific precipitation of the DNA by gluteraldehyde is shown in FIGS. 2G, H and I. Again the chromatin from the normal cells is less dispersed than that from the invasive cells.

The differential dispersive effect of proteinase K differs from the effects of some other proteolytic agents and protein binding modulators evaluated in that the effects of these other agents on chromatin from both normal and abnormal cells can be largely reversed by the addition of recondensation agents such as polyamine 11172 or polyamine 11158; histone H1; or topoisomerase IIa, or DNA precipitation agents such as glutaraldehyde or certain DNA binding dyes. The assays described herein may be used to identify other proteases that exhibit differential effects on chromatin stability for use in the screening and diagnostic methods described herein.

### Example 4

### Screening Drug Candidates

The method of Example 2 can be used to assess the effectiveness of anti-cancer drugs such as polyamines 11158 and 11172 in suppressing or blocking the invasive behavior of cancer cells. This effectiveness can be assessed by treating cancer cells with the drug; removing the chromatin from the cells as previously described; treating with 5 Units of ALU or DNAase in DMEM for 30 to 60 minutes; staining the samples with ethidium bromide or other DNA binding dye to facilitate visualization; and comparing the degree of chromatin degradation with that obtained by like treatment of chromatin isolated from cancer cells that have not been treated with the drug and from non-cancerous cells of the same type. The chromatin of invasive cancer cells is largely unaffected by this treatment while the chromatin of normal and non-invasive cells is largely degraded. Drug efficacy is evidenced by increased degradation of chromatin from drug treated cells relative to the degradation of chromatin from otherwise identical cells that have not been treated with the drug. The chromatin from normal cells of the same type serves as a control in this method.

### Example 5

### Identification of Chromatin Degrading Agents

The methods of the invention may be used to detect and identify drugs and other chemical and biological entities that differentially degrade the chromatin of cancerous cells relative to that of normal cells. Such agents may, by way of example, include, but are not limited to nucleases and endonucleases other than ALU and DNAase, caspases, catalytic RNA, and other materials. Chromatin strands from normal and cancerous cells of the same type are exposed to the agent being evaluated under identical conditions for a pre-selected period of time; stained with ethidium bromide or other DNA binding dye to facilitate visualization; compared to determine whether the chromatin from the cancerous or normal cell is preferentially degraded.

### Example 6

### Methods Using Cells

The methods of the previous embodiments may be applied to intact cells as well as to isolated chromatin strands. By way of example, intact melanoma cells grown on gelatin coated glass cover-slips as described above can be permeabilized by treatment with a 0.5% solution of the detergent Triton X-100 in DMEM for 2 minutes; washed with DMEM to remove residual detergent; and treated with 100 units of ALU in DMEM for one to three hours. This embodiment can be practiced using living or preserved cells adherent to a supporting substrate and to living and preserved cells that are suspended in a medium.

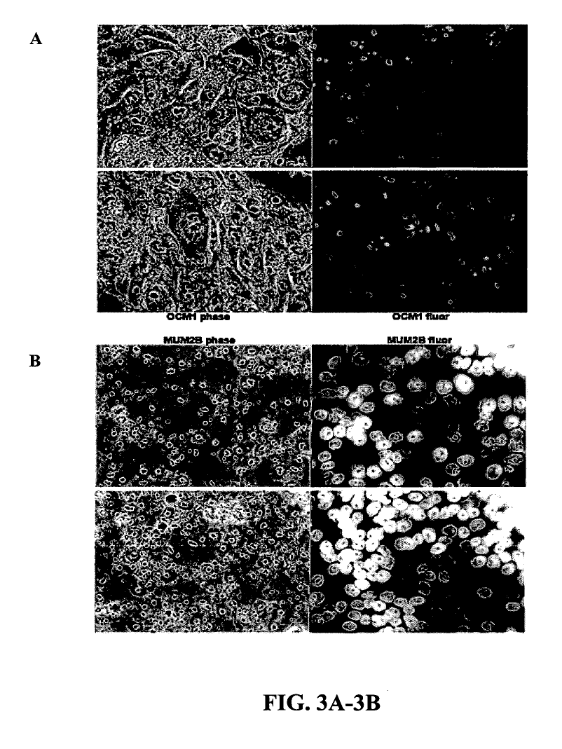

FIG. 3 illustrates the effects of this treatment upon non-invasive OCM-1a and highly invasive MUM-2B human melanoma cells. The phase contrast images in the left hand columns of FIGS. 3a and 3b show cultured interphase OCM-1a and MUM-2B cells, respectively, while the fluorescence images in the right hand columns of these Figures show the same cells after permeabilization with Triton X100; digestion with ALU; and staining with ethidium bromide.

In addition to being useful for the assessment of the invasiveness of cancer cells, this embodiment also finds utility in the screening of specimens for the presence of cancer cells due to the distinctive differences in the chromatin degradation patterns exhibited by normal and cancerous cells. A particular advantage of this application of the present invention is that it inherently discriminates between invasive cells and cells exhibiting benign reactive and repairative changes that often cause diagnostic errors in morphological and immunological tests.

The previously described embodiments are specific to the endonuclease ALU. However, the nuclease DNAase may be substituted for ALU in the present and the next described embodiments with some loss of specificity in the ability to differentiate between cells of differing degrees of invasiveness. Sufficient specificity remains, however, to permit the use of this nuclease for the differentiation between normal and cancerous cells.

### Example 7

### Methods Using Nuclei

Cells adherent to a 12 mm diameter glass cover-slip are prepared as described above. The cover slip with adhered cells is placed cell-side down in a 50 cc conical centrifuge tube containing 5 cc. of 10 mg/ml cytochalasin B in normal growth medium such that the edge of the cover-slip seats against the conical walls of the tube and the plane of the cover-slip is perpendicular to the long axis of the tube. Centrifugation results in the cell nuclei being displaced through the cell membranes and collecting as a pellet in the bottom of the centrifuge tube. The enucleated cells remain attached to the cover-slip.

The collected cell nuclei are washed in DMEM and permeabilized by treatment with a 0.1% solution of the detergent Triton X-100 in DMEM for two minutes before being treated with ALU or DNAase. As described above, ALU selectively degrades the chromatin in nuclei from normal and non-invasive cells. Furthermore, treatment of the permeabilized nuclei with 100 units of DNAase in DMEM for 30-60 minutes digests the chromatin of the nuclei from normal and non-invasive cells while leaving the chromatin in nuclei from invasive cells largely intact.

### Example 8

### MSP I Digestion

Methods

Human fibroblasts, poorly invasive human melanoma cells (OCM-1), and highly invasive human melanoma cells (MUM-2B) were scraped from their plastic cell culture flask bottoms with a rubber policeman to avoid disrupting their chromatin structure with EGTA, or their glycocalyces with trypsin. A 25 μL drop of each cell slurry was placed on a glass slide, and incubated for 30 minutes to an hour, until the drops-containing cells completely dried. Then, 0.5 μL of MSP I was added to a 25 μL drop of DMEM or PBS, and the 25 μL drop was placed onto the dried cell blots, and the slide was then placed in a 37° C. incubator in a sealed humidified chamber for 24 hours. After digestion, MSP I was removed and replaced with ethidium bromide, visualized under an epi-fluorescence microscope, and the blots were photographed.

Results

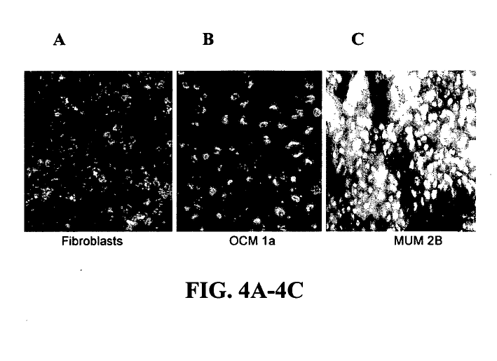

Whether incubated for 1, 2, 4, 5, 6, and 24 hours in MSP I, sequestration from digestion with MSP I appeared to increase with increasingly invasive cell behavior. Normal stromal cells, such as fibroblasts, were digested to a greater degree as compared to poorly invasive cells or to highly invasive cells. Cells are typically obtained mechanically, because trypsinization of cells in a trypsin-EGTA solution generated non-specific and sometimes completely refractory sensitivities to the enzyme(s). When trypsin EGTA was employed, for example, cell chromatin, regardless of the cell type, was much more stable to digestion with all restriction enzymes compared to mechanically isolated cells. A majority of the time cells demonstrated a gradation of sensitivity with normal cells being most sensitive, poorly invasive cells less sensitive, and highly invasive cell most refractory, if not completely refractory to digestion with MSP I, as well as other restriction enzymes (FIG. 4A-4C).

The scraping of cells from flasks, rather than EGTA-trypsinizing them from flasks, typically serves two purposes: 1) anticipation of the MSP 1 digestion being employed in a translational setting, in which proteases such as trypsin are normally not, and could not be used to obtain cells from a human patient, and 2) avoidance of the reagent, EGTA, which is commonly employed to accelerate cell dissociation from tissues or tissue culture flasks. In addition, the hypothesis that withdrawal of essential ions such as magnesium with EGTA or EDTA disrupts adhesion receptors specifically, has been shown to be simplistic, due to the fact that these chelators have profound effects upon chromatin organization and structure (Maniotis et al., 1997). Experiments employing EGTA-trypsin as the means of cell isolation have shown that sensitivities to restriction enzymes among different cell types is radically more stable to digestion, probably because of the effects of EGTA, and differences in cell aggregation due to trypsin-induced clumping. Therefore, to enhance potential clinical utility, and to avoid altering chromatin organization MSP I was employed without protease digestion or the presence of EGTA but instead, simply mechanically removed cells from their environment as they would be removed from a patient.

The fact that MSP I digestion could differentiate among nuclei of cells belonging to normal, poorly invasive and highly invasive cells, suggests that methylation of higher order structure is important, and may be the key factor in regulating a cell's cancerous or non-cancerous state, as well as a cell's pattern of methylation. Because digestion was cell type specific, rather than gene-type specific, the assay potentially can discriminate between normal, lowly and highly invasive cells, from sporadic tumors (99%) where linkage groups are unknown, and which familial linkage is not established, as well as the familial tumors (1%) where suspected oncogenes (p53, p21, retinoblastoma (rb), and the like) are thought to play some causative role.

### Example: 9

### Detection of Invasive Cells by Flow Cytometry

The methods described in the preceding examples of the present invention for the detection of invasive cells require that the cells are in contact with a substrate, typically a layer of absorbed protein, prior to the treatment of the cells with a chromatin-degrading agent. This step can be inconvenient in a clinical setting. The cells of hematological cancers are typically collected as suspensions of cells in a fluid medium such as blood or lymphatic fluid. Similarly, certain methods such as fine needle aspiration (FNA) that are in common clinical use for the initial collection of the specimens from solid tumors result in the formation of a suspension of the collected cells in a fluid medium. Furthermore, the dispersion of cells into a fluid medium is an intrinsic element in the process of preparing monolayer preparations on microscope slides and in preparing specimens for evaluation by tissue culture and similar methods. For this reason it is convenient to be able to practice the present invention in a manner that directly utilizes specimen cells in fluid suspension rather than requiring that the cells be first be transferred to a solid substrate, such as a glass cover slip. The utilization of suspended cells in the practice of this invention in a manner that is directly analogous to the methods described above.

Cultured cells of differing degrees of invasiveness are utilized for illustrative purposes in this example. Suspensions of cells from patient specimens may similarly be employed. The cultured cell lines employed in this example are: WI-38 fibroblasts (normal cells); OCM1 (a poorly invasive a primary uveal melanoma); M619 (a highly invasive primary uveal melanoma; and MUM2B (a highly invasive metastatic uveal melanoma). All cells were grown in monolayer culture according to well-known standard methods; mechanically harvested into DMEM medium and pelleted by centrifugation at 1400 RPM for 5 minutes in a desktop centrifuge.

The cellular pellet was re-suspended in 0.1% Triton X-100; incubated for 1 minute at room temperature; spun down again at 1400 rpm for 5 minutes; and resuspended in DMEM. Propidium iodide (PI; 10 μl/ml; Molecular Probes, Eugene, Oreg.) was added to an aliquot of this suspension. 0.5 μl of Alu I restriction enzyme in 40 μl of DMEM was added to the remaining cell suspension and the preparation was incubated at 37° C. Aliquots of this mixture were taken for evaluation at 0 (baseline), 1, 3, and 5 hours after the addition of ALU. Propidium iodide (10 μl/ml; Molecular Probes, Eugene, Oreg.) was added to each of these digested samples. The resulting digested and stained cell suspensions were analyzed according to standard methods using a FACS Calibur flow cytometer (BD Bioscience, San Jose, Calif.) equipped with 488 nm laser excitation, detectors for forward and side scatter, and 520, 575, and 675 nm detectors for fluorescence signals. 10,000 cells were counted and the results were analyzed with FACS dot-plots and histograms. CellQuest software (BD Bioscience) was used for statistical analyses.

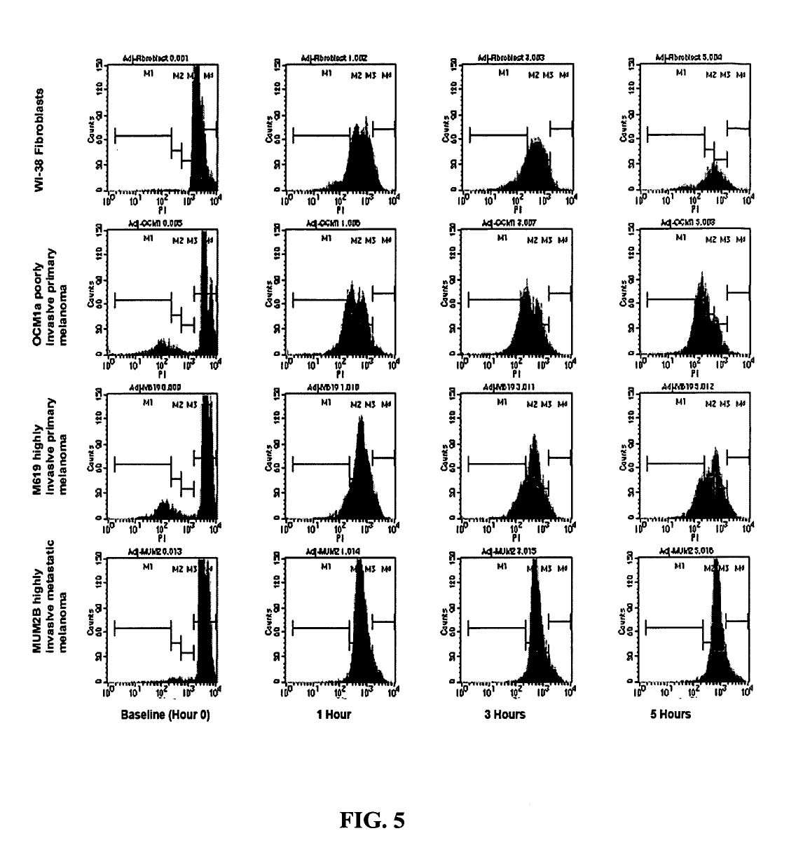

Propidium iodide (PI) is a stoichiometric DNA fluorescent staining agent thus allowing the DNA content of the cells being evaluated to be determined from the fluorescent signal intensity as measured by flow cytometry. The aliquot of cell suspension that was treated with PI after permeabilization, but not digested with ALU serves as a reference for the amount of DNA present in each of the cell preparations prior to the start of treatment. FIG. 5 shows the flow cytometer fluorescence intensity histogram plots measured for each cell line at 1, 3 and, 5 hours exposure to Alu I restriction enzyme followed by staining with PI.

A reduction in PI signal for WI-38 fibroblasts relative to the undigested control was detected at one hour, with further decreases at 3 and 5 hours. By five hours, a significant component of the baseline signal decreased below the limits of the instrument's detection threshold (FIG. 5, top row). This indicates a significant degradation of the DNA in normal fibroblasts. Poorly invasive OCM1a melanoma cells exhibited a similar reduction in the PI signal after 1 hour Alu I enzyme digestion, but thereafter, the signal intensity did not decrease significantly (FIG. 5, 2nd row).

Unlike the WI-38 fibroblasts and the poorly invasive OCM1a melanoma cells, highly invasive M619 or MUM2B melanoma cells showed no significant loss of PI signal at one hour Alu I digestion (FIG. 5, bottom two rows). However, the PI signal from the highly invasive primary M619 melanoma cells had decreased by 3 hours, while the signal for the highly invasive metastatic MUM2B melanoma cells was not significantly different from the baseline signal even at 5 hours. Therefore, on the basis of measuring the PI signal after exposure of the permeabilized cells to Alu I restriction enzyme for different periods of time, it is possible to discriminate between each of the four cell lines and thereby to utilize flow cytometry for the detection and classification of invasive cells.

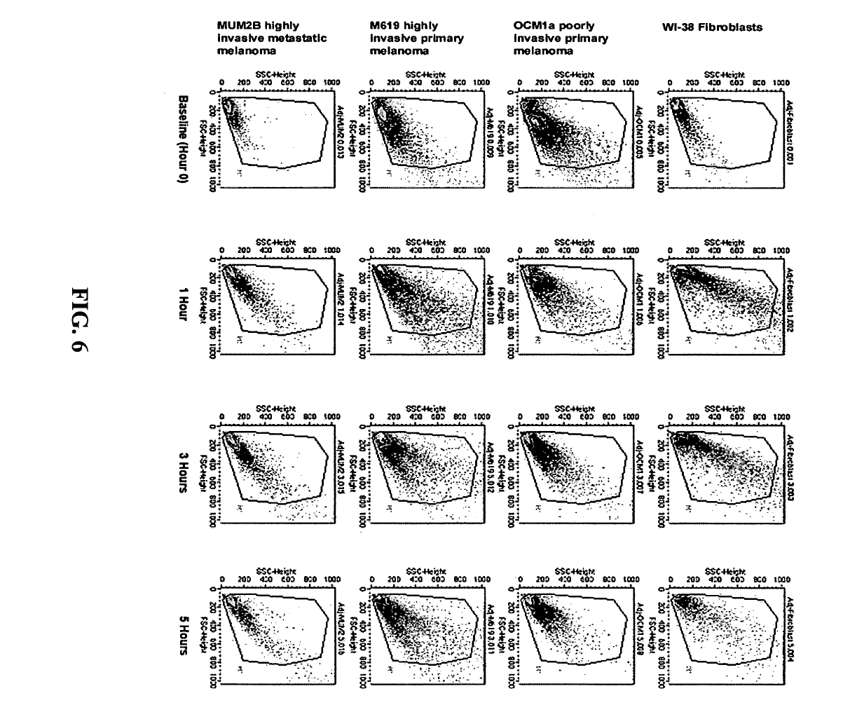

Forward scatter (representing cell size), and side scatter (representing internal cellular complexity) were also measured before exposure to Alu I and after 1, 3, and 5 hours digestion (FIG. 6) for each of the four cell lines. Significant changes in forward and side scatter were detected in WI-38 fibroblasts after exposure to Alu I (FIG. 6, top row). At 1 hour digestion, the forward scatter is dramatically decreased, while the side scatter is increased. These changes in cell size and internal complexity progressed through 3 and 5 hours. By 5 hours, the number of detectable cells had decreased significantly indicating extensive digestion of the DNA. A modest reduction in forward scatter and increase in side scatter was detected a 1 hour in the OCM1a and MUM2B cells with no significant additional changes in these parameters at 3 and 5 hours. By contrast, there was no significant change detected in MUM2B cells in either forward or side scatter at any time point after exposure to Alu I restriction enzyme. Therefore, changes PI signal, forward scatter, and side scatter relative to the undigested baseline reference samples over a 5 hour Alu I restriction enzyme digest, demonstrate that it is possible to objectively classify WI-38 normal fibroblasts, OCM1a low invasive primary melanoma cells, M619 highly invasive primary melanoma cells, and MUM2B highly invasive metastatic melanoma cells by flow cytometry in accordance with the present invention.

### Example: 10

### Drug Evaluation Using Nuclei

Cells adherent to a 12 mm diameter glass cover-slip are prepared as described herein. The cover slip with adhered cells is placed cell-side down in a 50 cc conical centrifuge tube containing 5 cc of 10 mg/ml cytochalasin B in normal growth medium such that the edge of the cover-slip seats against the conical walls of the tube and the plane of the cover-slip is perpendicular to the long axis of the tube. Centrifugation at 1400 RPM for 5 minutes results in the cell nuclei being displaced through the cell membranes and collecting as a pellet in the bottom of the centrifuge tube.

The collected cell nuclei are washed in DMEM and permeabilized by treatment with a 0.1% solution of the detergent Triton X-100 in DMEM for two minutes before being treated with ALU or DNAase. As described above, ALU selectively degrades the chromatin in nuclei from normal and non-invasive cells. Furthermore, treatment of the permeabilized nuclei with 100 units of DNAase in DMEM for 30-60 minutes digests the chromatin of the nuclei from normal and non-invasive cells while leaving the chromatin in nuclei from invasive cells largely intact. The scatter data presented in example 15 suggests that degradation of the chromatin in a cell nucleus correlates with and is possibly causally related to changes in the cytoplasmic structure of the cell that occur over a period of minutes to an hour. Other data related to example 13, but not herein described suggest that changes in the cytoskeleton which may be detectable by light scattering can influence the state of the chromatin in the cell nucleus. The displacement of the nucleus through the cell membrane that occurs in the present method appears to be sufficiently rapid as to permit differentiation between nuclear and cytoplasmic factors without significant levels of confounding interactive effects.

The descriptions of particular embodiments above are intended to be representative of and not limiting to the present invention. Additional embodiments of the invention are within the scope and spirit of the claims.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

The following references, to the extent that they provide exemplary procedural or other details supplementary to those set forth herein, are specifically incorporated herein by reference.
U.S. Provisional Patent application 60/476,580, filed on Jun. 6, 2003;
U.S. Provisional Patent application 60/511,543, filed Oct. 14, 2003
U.S. Provisional Patent application 60/526,792, filed Dec. 4, 2003
U.S. Provisional Patent applications 60/______, filed May 26, 2004
Bojanowski et al., Experimental Cell Research, Vol. 244, pp. 286-294, 1998
Bojanowski, et al., Journal of Cellular Biochemistry, Vol. 69, pp. 127-142, 1998.
Chen et al., Invest Ophthalmol Vis Sci. 44(7):2834-40, 2003.
Current Protocols in Cell Biology, John Wiley & Sons, Edited by: Juan S. Bonifacino, Mary Dasso, Jennifer Lippincott-Schwartz, Joe B. Harford, and Kenneth M. Yamada, 2001.
Current Protocols in Cytometry, John Wiley & Sons, Edited by Robinson et al., 1997.
Freshney, Culture of Animal Cells and Manual of Basic Techniques. 2nd edition, New York, 1987.
Garinis et al., J Pathol. 198(4):442-49, 2002.
Hughes et al., Cell Death and Differentiation, Vol. 4, pp. 200-208, 1997.
Maniotis et al., Journal of Cellular Biochemistry, Vol.65, pp.114-130, 1997.
Molecular Probes: Handbook, updated Sep. 7, 2003, probes.com/handbook
Murphy, Fundamentals of Light Microscopy and Electronic Imaging, Wiley-Liss, Inc. 2001
Pratt, Digital Image Processing: PIKS Inside, 3rd Edition, John Wiley & Sons, 2001.
Puck et al., International Reviews of Cytology, Vol. 132, pp. 75-108, 1992.
Suter et al., The EMBO Journal, Vol. 16, pp. 2150-2160, 1997.

1. A method for assessing the invasive potential of a cell comprising:
a) contacting chromatin of a cell with one or more chromatin modifying agents; and
b) evaluating chromatin stability by assessing chromatin degradation.

2. The method of claim 1, further comprising isolating nuclei from the cells prior to contacting the chromatin with one or more chromatin modifying agents.

3. The method of claim 1, further comprising isolating the chromatin from the cells prior to contacting the chromatin with one or more chromatin modifying agents.

4. The method of claim 1, wherein the cell and nuclear membrane is permeabilized.

5. The method of claim 2, wherein nuclear membranes are permeabilized.

6. The method of claim 1, wherein the chromatin modifying agent is a proteolytic enzyme.

7. The method of claim 6, wherein the proteolytic enzyme is proteinase K.

8. The method of claim 6, wherein the chromatin is re-aggregated after contact with the chromatin modifying agent.

9. The method of claim 8, wherein chromatin re-aggregation is initiated by contacting the chromatin with a DNA binding dye, a poly-amine, a histone, a topoisomerase, or glutaraldehyde prior to assessing chromatin stability.

10. The method of claim 1, wherein the chromatin modifying agent is a nuclease.

11. The method of claim 10, wherein the nuclease is DNAase.

12. The method of claim 10, wherein the nuclease is an endonuclease.

13. The method claim 12, wherein the endonuclease is ALU or MSP 1.

14. The method of claim 1, wherein evaluation of the chromatin is on a surface that is planar or approximately planar.

15. The method of claim 1, wherein evaluation of the chromatin is in a suspension in a fluid medium.

16. The method of claim 15, wherein the evaluation is by flow cytometry.

17. The method of claim 1, wherein chromatin degradation is evaluated qualitatively.

18. The method of claim 1, wherein the chromatin degradation is evaluated quantitatively.

19. The method of claim 1, wherein chromatin degradation is evaluated by visual microscopy, image analysis, or flow cytometry.

20. The method of claim 1, wherein optical contrast of the chromatin is enhanced by contacting the chromatin with a DNA binding dye prior to evaluation.

21. The method of claim 20, wherein the DNA binding dye comprises a chromatic dye.

22. The method of claim 20, wherein the DNA binding dye comprises a fluorescent dye.

23. The method of claim 22, wherein the fluorescent dye is ethidium bromide, acridine orange, TO-PRO, YO-YO, YO-PRO or PO-PRO.

24. A method for assessing the effectiveness of a candidate therapeutic agent comprising:
a) contacting a cell with the candidate therapeutic agent;
b) contacting the chromatin of the cell with a chromatin modifying agent;
c) evaluating chromosome stability by assessing chromatin degradation; and
d) assessing the effectiveness of the candidate therapeutic agent by comparing chromatin degradation resulting from treatment of the cell with the candidate therapeutic agent with a cell not treated with the therapeutic agent.

25. The method of claim 24, further comprising isolating chromatin from the cell prior to contacting the chromatin with the chromatin modifying agent.

26. The method of claim 24, wherein the cell is a hyperproliferative cell.

27. The method of claim 26, wherein the hyperproliferative cell is a cancer cell.

28. The method of claim 24, further comprising isolating nuclei from the cells prior to contacting chromatin with a chromatin modifying agent.

29. The method of claim 24, further comprising isolating the chromatin from the nuclei of the cells.

30. The method of claim 24, wherein the cell and nuclear membranes are permeabilized.

31. The method of claim 24, wherein the chromatin modifying agent is a proteolytic enzyme.

32. The method of claim 31, wherein the proteolytic enzyme is proteinase K.

33. The method of claim 31, wherein chromatin is re-aggregated before evaluating chromatin stability.

34. The method of claim 33, wherein chromatin re-aggregation is initiated by contacting the chromatin with a DNA binding dye, a poly-amine, a histone, a topoisomerase, or glutaraldehyde prior to evaluation of chromatin stability.

35. The method of claim 24, wherein the chromatin modifying agent is a nuclease.

36. The method of claim 35, wherein the nuclease is DNAase.

37. The method of claim 35, wherein the nuclease is an endonuclease.

38. The method claim 37, wherein the endonuclease is ALU or MSP 1.

39. The method of claim 24, wherein evaluation of chromatin stability is performed on a surface that is planar or approximately planar.

40. The method of claim 24, wherein evaluation of chromatin stability is performed as a cell suspension in a fluid medium.

41. The method of claim 40, wherein the evaluation is by flow cytometry.

42. The method of claim 24, wherein the chromatin degradation is evaluated qualitatively.

43. The method of claim 24, wherein the chromatin degradation is evaluated quantitatively.

44. The method of claim 24, wherein chromatin degradation is evaluated by visual microscopy, image analysis, flow cytometry.

45. The method of claim 24, wherein optical contrast of the chromatin is enhanced by contacting the chromatin with a DNA binding dye prior to evaluation.

46. The method of claim 45, wherein the DNA binding dye comprises a chromatic dye.

47. The method of claim 45, wherein the DNA binding dye comprises a fluorescent dye.

48. The method of claim 47, wherein the fluorescent dye is ethidium bromide, acridine orange, TO-PRO, YO-YO, YO-PRO or PO-PRO.

49. A method comprising differentiating between normal and invasive cancerous cells by evaluating the degree of chromatin degradation by a nuclease.

50. The method of claim 49, wherein the nuclease is DNAase.

51. The method of claim 49, wherein the nuclease is an endonuclease.

52. The method of claim 51, wherein the endonuclease is ALU or MSP 1.

53. The method of claim 49, further comprising isolating nuclei from the cells prior to contacting the chromatin with one or more chromatin modifying agents.

54. The method of claim 53, further comprising isolating the chromatin from the nuclei of the cells prior to contacting the chromatin with one or more chromatin modifying agents.

55. The method of claim 49, wherein the cell and nuclear membranes are permeabilized.

56. The method of claim 49, wherein the chromatin is re-aggregated prior to evaluating chromatin degradation.

57. The method of claim 56, wherein chromatin re-aggregation is initiated by contacting the chromatin with a DNA binding dye, a poly-amine, a histone, a topoisomerase, or glutaraldehyde prior to assessing chromatin stability.

58. The method of claim 49, wherein evaluation of the chromatin is on a surface that is planar or approximately planar.

59. The method of claim 49, wherein evaluation of the chromatin is as a suspension in a fluid medium.

60. The method of claim 59, wherein the evaluation is by flow cytometry.

61. The method of claim 49, wherein chromatin degradation is evaluated qualitatively.

62. The method of claim 49, wherein the chromatin degradation is evaluated quantitatively.

63. The method of claim 49, wherein chromatin degradation is evaluated by visual microscopy, image analysis, or flow cytometry.

64. The method of claim 49, wherein optical contrast of the chromatin is enhanced by contacting the chromatin with a DNA binding dye prior to evaluation.

65. The method of claim 64, wherein the DNA binding dye comprises a chromatic dye.

66. The method of claim 64, wherein the DNA binding dye comprises a fluorescent dye.

67. The method of claim 66, wherein the fluorescent dye is ethidium bromide, acridine orange, TO-PRO, YO-YO, YO-PRO or PO-PRO.
